(19) United States
(12) Patent Application Publication
(10) Pub. No.: US 2008/0208174 A1
(43) Pub. Date: Aug. 28, 2008

(54) Device for the Controlled Release of a Predefined Quantity of a Substance

(75) Inventors: Mark Thomas Johnson, Eindhoven (NL); Ralph Kurt, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/994,418

(22) PCT Filed: Jun. 29, 2006
(86) PCT No.: PCT/IB2006/052174

§ 371 Date: Jan. 2, 2008

(30) Foreign Application Priority Data
Jul. 5, 2005 (EP) 05106081.2

(51) Int. Cl. A61K 9/22; A61M 31/00

(52) U.S. Cl. 604/891.1; 607/88; 604/288.01

(57) ABSTRACT

The application provides a device (10) for the controlled release of a predefined quantity of a substance from a compartment (20). The device comprises a matrix arrangement of compartments in a substrate, each compartment being closed by at least one release mechanism, at least one first electrode (40) and at least one second electrode (50) being assigned to each compartment, the device comprising a plurality of selection lines (60) and a plurality of signal lines (70), the number of compartments exceeding the sum of the number of selection lines and the number of signal lines, each first electrode or each second electrode being electrically connected via at least one active component to one of the plurality of selection lines and/or to one of the plurality of signal lines.

The present invention relates a device for the controlled release of a predefined quantity of a substance. The present invention further relates to a method for controllably releasing a predefined quantity of a substance from a compartment.

Accurate delivery of small, precise quantities of one or more chemicals into a carrier fluid are of great importance in many different fields of science and industry. Examples in medicine include the delivery of drugs to patients using intravenous methods, by pulmonary or inhalation methods or by the release of drugs from vascular stent devices. Examples in diagnostics include releasing reactions into fluids to conduct DNA or genetic analysis, combinatorial chemistry, or the detection of a specific molecule in an environmental sample. Other applications involving the delivery of chemicals into a carrier fluid include the release of fragrances and therapeutic aromas from devices into air and the release of flavoring agents into a liquid to produce beverage products.

Devices for the controlled release of a predefined quantity of a substance are generally known. For example, the US patent application US 2004/0034332 A1 discloses an implantable device for controlled delivery of a drug, the device including a microchip which have reservoirs containing the molecules for release. The microchip device includes a substrate, at least two reservoirs in the substrate containing the molecules for release and a reservoir cap positioned on or within a portion of the reservoir and over the molecules, so that the molecules are controllably released from the device by diffusion through or upon disintegration or rupture of the reservoir caps. Each of the reservoirs of a single microchip can contain different molecules which can be released independently. One drawback of the known device is that each reservoir is directly contacted to an electrode which is used to electrically break the seal layer or the cap by applying a current and to release the drug. A weakness of the prior art system is that one external electrical connection is required for each compartment or for each reservoir from which the drug is to be released. This strongly limits the number of compartments, which can be realized on a single device as the space required for all the electrical connections becomes prohibitive.

It is therefore an object of the present invention to provide a device for the controlled release of a predefined quantity of a substance that has an increased number of reservoirs or compartments without the need for providing one external electrical connection for each compartment to be controlled independently.

The above object is achieved by a device and a method for the controlled release of a predefined quantity of a substance according to the present invention. The device for the controlled release of a predefined quantity of a substance comprises a matrix arrangement of compartments in a substrate, each compartment being closed by at least one release mechanism, at least one first electrode and at least one second electrode being assigned to each compartment, the device comprising a plurality of selection lines and a plurality of signal lines, the number of compartments exceeding the sum of the number of selection lines and the number of signal lines, each first electrode or each second electrode being electrically connected via at least one active component to one of the plurality of selection lines and/or to one of the plurality of signal lines.

An advantage of the apparatus according to the invention is that it is possible to realize a controlled substance or drug delivery system based upon a multiplicity of individual drug release compartments where the number of compartments is very high, i.e. in the range of 100-1,000,000 compartments. According to the prior art, the number of compartments is strongly limited by the need to contact each compartment individually by a connecting line.

A further advantage of the present invention is that the control of delivery of a substance or a drug is based upon an active matrix principle. This is in contrast to the prior art systems where each compartment is directly connected to an electrical connection. By the use of an active matrix, it is feasible to release drugs from any of the large number of compartments of the order of 100-1,000,000 in a controlled manner. This is not feasible if every compartment were to be individually controlled by a dedicated control device as the costs and space required to incorporate such a control system would be prohibitive. A further advantage of the present invention is that thereby, applications such as for example external drug delivery systems (patches), implantable drug delivery systems or oral drug delivery systems (e-pill) are possible. A drug delivery system according to the present invention maybe applied for delivery of a single drug, but can be advantageously applied to a system where several different drugs are applied from the same array or the same device. An active matrix type device for the controlled release of a predefined quantity of a substance is realized by electrically connecting each compartment or at least each release mechanism of a compartment or at least two electrodes associated or assigned to a compartment via at least one active component to one of a plurality of selection lines and/or to one of a plurality of signal lines. The active matrix principle is realized by connecting at least one of the electrodes (first or second electrode assigned to each compartment) to the selection lines and/or the signal lines via an active electrical or electronic component. Such active components include especially transistors like switch transistors (FET-transistors (field effect transistors) and/or bipolar transistors).

In a preferred embodiment of the present invention, the release mechanism is a one-time release mechanism. This means that the release mechanism is in some manner “destroyed” by applying a release signal above the threshold and the release mechanism is not re-usable. Thereby, it is possible to provide the release mechanism very cost-effectively and easy to manufacture. Nevertheless, the present invention also refers to a release mechanism which is closable once it has been opened (for the first time) and further on re-openable at least a second time. Such an embodiment employing a re-closable and re-openable release mechanism is less preferred because this usually implies higher costs.

Highly preferably, the release mechanism according to the present invention is provided by means of a closure cap. A closure cap is one specific and preferred embodiment of realizing a release mechanism. Examples of other release mechanisms are: a polymer membrane or a gel that releases drugs if heated (decomposition of a carrier matrix or changing properties of it, such as breaking dedicated chemical bonds) or membranes that change their permeability for certain molecules upon applying an electrical potential.

In a preferred embodiment of the present invention, each compartment is defined by means of one specific selection line out of the plurality of selection lines and one specific signal line out of the plurality of signal lines. As a result, the matrix principle for addressing an individual compartment is realized and therefore the number of connection lines strongly reduced.

In a further preferred embodiment, the number of compartments is in the order of magnitude of the number of selection lines multiplied by the number of signal lines. It is therefore possible to reduce the required connecting line on the device even more and therefore render the device smaller, of lighter weight and more cost-effective.

In a further preferred embodiment, the number of selection lines is substantially the same as the number of signal lines. It is therefore possible to further reduce the required connecting lines on a device with a given number of compartments and therefore render the device even smaller, of lighter weight and even more cost-effective.

In a still further preferred embodiment of the present invention, the active component comprises a transistor assigned to each compartment. In an alternatively preferred embodiment of the present invention, the active component comprises a first transistor and a second transistor. An advantage of using a transistor or transistors as active components in an inventive device is that it is possible to render the inventive device cost-effective and still relatively small because it is possible to realize transistors on very small surface areas of, e.g., a glass substrate. According to the invention, the use of an active component, especially one or a plurality of transistors provides an enhanced specificity in selecting a compartment compared to directly connecting one or both of the first and second electrode to the selection and/or signal lines. The use of one transistor as an active component aims at reducing relatively the required size (e.g. needed surface area) of a compartment. The use of at least a first and a second transistor aims at enhancing the functionality of driving the compartment (e.g. current and/or voltage controlled drug release) or at enhancing the functionality of the device (e.g. including further functions at each compartment like memorizing whether the drug release has already occurred or not).

It is much preferred according to the present invention to use a thin film transistor as the transistor or as the transistors of the active component of each compartment of the device. This renders the device more cost-effective and it is possible to use lighter materials.

In a further embodiment of the invention the active element comprises a diode assigned to each compartment. An advantage of using a diode or diodes as active components in an inventive device is that it is possible to render the inventive device even more cost-effective and still relatively small, because it is possible to realize diodes on very small surface areas of, e.g., a glass substrate in a technology that is more cost-effective than a transistor-based technology.

In a further embodiment of the invention the active element comprises a non-linear resistance element, specifically a metal-insulator-metal (MIM) diode assigned to each compartment. An advantage of using a MIM diode or MIM diodes as active components in an inventive device is that it is possible to render the inventive device even more cost-effective and still relatively small because it is possible to realize MIM diodes on very small surface areas of, e.g., a glass substrate in a technology that is more cost-effective than a transistor-based technology.

In a further preferred embodiment, the active component comprises a memory means. This is advantageous for providing an enhanced control possibility of the functionality of the inventive device.

In a still further preferred embodiment of the present invention, a first group of compartments is provided to contain a first quantity of a first substance and a second group of compartments is provided to contain a second quantity of a second substance. An advantage of the device according to the present invention is that a very flexible substance release mechanism can be implemented in the structure of the inventive device. For example, it is possible to provide compartments of different size, thereby being able to contain different volumes of the substance or substances to release. For example, if at a given moment a greater quantity of a substance is to be released, a device can be controlled accordingly and open a compartment having an appropriate size and hence an appropriate volume of the substance to be released. This is instead of releasing the same quantity of substance from a certain number of smaller compartments which would have the same effect. Of course, the release of an appropriate quantity of a substance out of one single compartment is easier to control and therefore makes the device according to the present invention smaller, of lighter weight and more cost effective. Accordingly, the first and second substance can be different or identical. Another way to improve the flexibility of releasing substances like drugs or the like is to provide several different substances or different mixtures of substances in different compartments on the device, the different compartments being of the same or of a different size. It is thereby possible to controllably release for example two different drugs alternatively during the day or during another time interval to the patient. Alternatively, it is also possible to further enhance the flexibility of use of the inventive device for example by providing differently sized compartments as well as different substances in the differently sized compartments. It is preferred according the present invention, that the first quantity is approximately half of the second quantity. It is thereby also possible to have a first group of compartments having a first volume or containing a first quantity of a substance, a second group of compartments containing each twice the first quantity, a third group containing four times the first quantity and a fourth group of compartments containing eight times of the first quantity. Thereby flexibility of releasing one or more substances is even further enhanced.

In a preferred embodiment of the present invention, the release mechanism of the compartment is provided removable or disintegratable by applying an electrical potential between the first electrode and the second electrode. It is then possible to very easily and quickly control the release of the substance out of one of the compartments.

In a further embodiment the first electrode and the second electrode of each compartment are provided electrically insulated from each other.

It is further preferred that the release mechanism is activated by means of an electro-chemical reaction or by means of heating the release mechanism, preferably by means of an electrical current. The device can be produced in a very cost-effective manner and the release of the substance can be made quicker and more accurate.

Further embodiments of the present invention are provided with a control unit for controlling the release of the substance. It is further preferred, that the number of compartments is at least 100, preferably at least 1,000, more preferably at least 10,000, still preferably at least 100,000 and most preferably at least 1,000,000 compartments.

The present invention also includes a method for controllably releasing a predefined quantity of a substance from a compartment, using a device comprising a matrix arrangement of compartments in a substrate, each compartment being closed by at least one release mechanism, at least one first electrode and at least one second electrode being assigned to each compartment, the device comprising a plurality of selection lines and a plurality of signal lines, the number of compartments exceeding the sum of the number of selection lines and the number of signal lines, the method comprising the steps of:

electrically connecting each first electrode or each second electrode via at least one active component to one of the plurality of selection lines and/or to one of the plurality of signal lines,

activating the active component and thereby applying an electrical potential or a current between the first electrode and the second electrode.

It is thereby possible to controllably release a specific quantity of a substance in a very rapid and easily controlled manner.

In a preferred embodiment of the method according to the present invention, more than one compartment release the substance at the same time. This may mean that more than one compartment are opened simultaneously and that the period of releasing the substance or the drug is then common for each of these compartments. Alternatively, it is also possible that a plurality of compartments are opened sequentially such that their period of release (usually much longer than the time required for opening a specific compartment) overlap and a release of the substance by more than one compartments is possible. It is thereby possible to very flexibly control the release of a substance.

These and other characteristics, features and advantages of the present invention will become apparent from the following detailed description, taken in conjunction with the accompanying drawings, which illustrate, by way of example, the principles of the invention. The description is given for the sake of example only, without limiting the scope of the invention. The reference figures quoted below refer to the attached drawings.

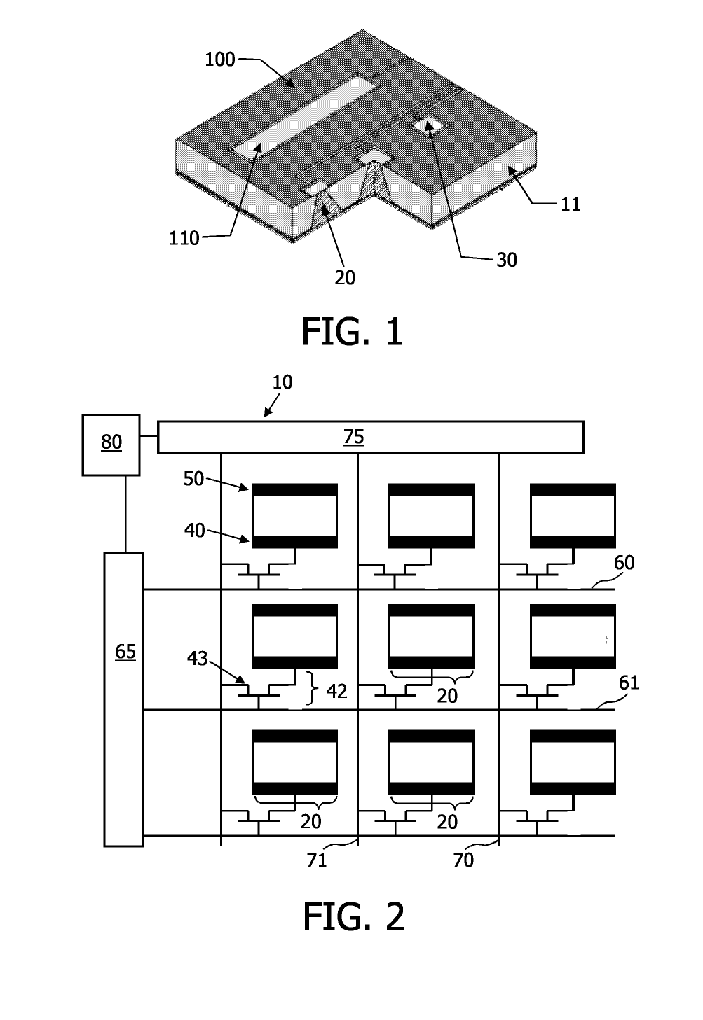

FIG. 1 illustrates schematically a device 100 according to the prior art showing a basic structure of a device of such a type.

FIG. 2 illustrates schematically a device according to the present invention.

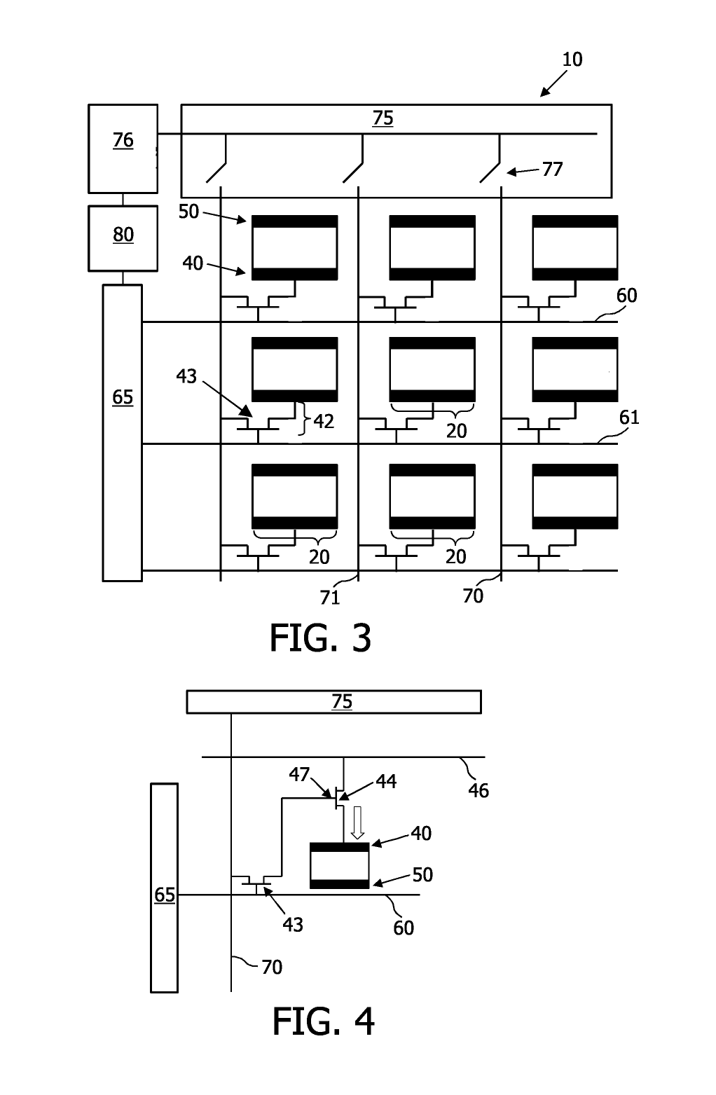

FIG. 3 illustrates schematically a device according to a further embodiment.

FIG. 4 illustrates schematically an embodiment of a compartment with an active component.

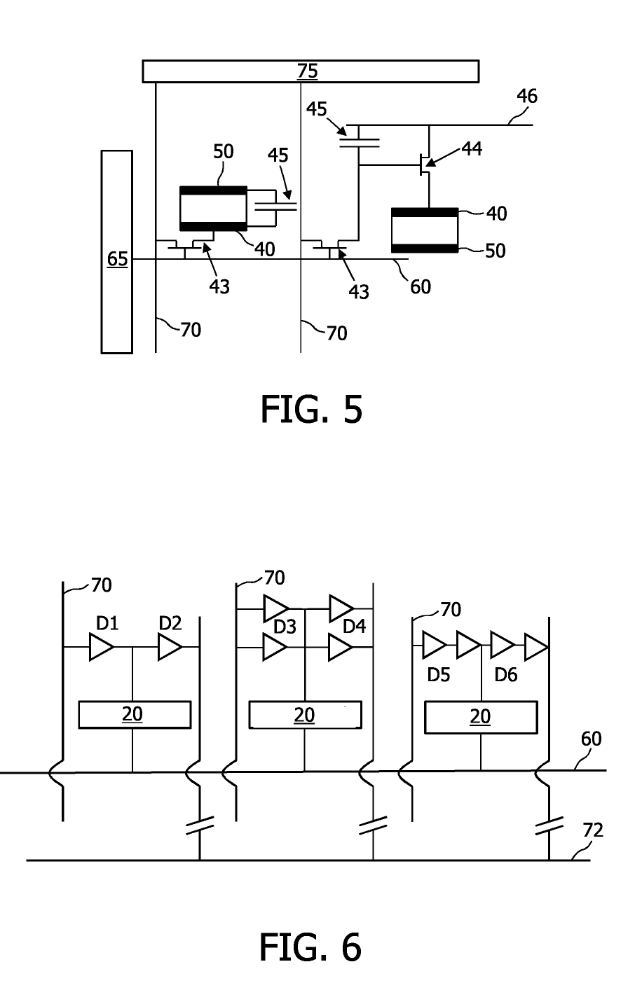

FIGS. 5 to 7 illustrate schematically further embodiments of compartments with active components.

FIG. 8 illustrates four different arrangements of compartments in a device according to the present invention.

The present invention will be described with respect to particular embodiments and with reference to certain drawings, but the invention is not limited thereto but only by the claims. The drawings described are only schematic and are non-limiting. In the drawings, the size of some of the elements may be exaggerated and not drawn to scale for illustrative purposes.

Where an indefinite or definite article is used before a singular noun, e.g. “a”, “an”, “the”, this includes a plural of that noun, unless otherwise specifically stated.

Furthermore, the terms first, second, third and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising”, used in the present description and claims, should not be interpreted as being restricted to the means listed thereafter; it does not exclude other elements or steps. Thus, the scope of the expression “a device comprising means A and B” should not be limited to devices consisting of only components A and B. It means that with respect to the present invention, the only relevant components of the device are A and B.

In FIG. 1, a known device 100 according to the prior art is schematically shown. The known device 100 comprises a substrate 11 where a plurality of compartments 20 are located. The compartments 20 are closed by a release mechanism 30, especially a closure cap 30. It can further be seen from FIG. 1 that there are electrode lines running to each of the compartments 20 or at least to or near to each of the release mechanisms 30. The connecting lines are not described with a reference sign in FIG. 1. The known device 100 further comprises an electrode area 110.

In FIG. 2 an inventive device 10 is schematically shown comprising a plurality of compartments 20 where only nine compartments 20 are shown. The device 10 comprises the compartments 20 in a substrate 11 comparable to the prior art devices. The substrate 11 is the structural body in which the compartments 20 are formed, e.g. it contains the etched, machined or molded compartments 20. A compartment 20 (which is also called a reservoir in the following) is a container for a substance. Micro-electromechanical system methods, micro-molding and micro-machining techniques known in the art can be used to fabricate the substrate 11 together with the compartments 20 from a variety of materials. Examples of suitable substrate materials include glass, metals, ceramics, semiconductors, degradable and non-degradable polymers. Preferably, the substrates are the well-known substrates for active matrix production of e.g. liquid crystal displays, namely glass, plastic films or metals. Bio-compatibility of the substrate material typically is preferred for in-vitro device applications. The substrate, or portions thereof, may be coated, encapsulated, or otherwise contained in a bio-compatible material before use. The substrate 11 can be flexible or rigid. In one embodiment, the substrate 11 serves as a support for a microchip device. In one example, the substrate 11 is formed of silicon. The substrate 11 can have a variety of shapes for shaped surfaces. It can, for example, have a release side, i.e. an area having release mechanisms, that is planar or curved. The substrate may for example be in a shape selected from discs, cylinders, or spheres. In one embodiment, the release side can be shaped to conform to a curved tissue surface. This would be particularly advantageous for local delivery of a therapeutic agent to that tissue surface. In another embodiment the backside (distal to the release side) is shaped to conform to an attachment surface. The substrate may consist of only one material or may be a composite or multi-laminate material, that is, composed of several layers of the same or different substrate materials that are bonded together.

In the schematical illustration of FIG. 2 of the inventive device 10, the inventive device 10 comprises for each compartment 20 a first electrode 40 and a second electrode 50. Here, the first and the second electrodes 40, 50 are not directly electrically connected, i.e. they are substantially insulated from each other by e.g. a dielectric medium such as a fluid. This means, that the electrical resistance created by materials separating the first and second electrode 40, 50 from each other are from a sufficiently high resistivity that regarding the applied voltages or potential differences there is no substantial current flow between the first and second electrode 40, 50. The inventive device 10 further comprises the compartments 20 in the form of a matrix arrangement. Further, the inventive device 10 comprises a plurality of selection lines 60 and a number of signal lines 70. Here, the select and signal lines are shown in a mutually perpendicular alignment of rows and columns, while other matrix arrangements, such as on a hexagonal or triangular grid would also be possible, providing the select and signal lines are configured in mutually different orientations. For the sake of example, one specific selection line 61 from the plurality of selection lines 60 is specifically shown in FIG. 2. Accordingly, one specific signal line 71 from the plurality of signal lines 70 is shown in FIG. 2. The specific selection line 61 and signal line 71 of FIG. 2 define the compartment 20 in the middle of the matrix arrangement of nine compartments 20 shown in FIG. 2. This means, that by selecting the specific selection and signal lines 61, 71, the compartment in the middle of the matrix arrangement is selected for being activated. The activation of the corresponding compartment is done by means of an active component which is generally referred to by reference sign 42. In the example of the inventive device shown in FIG. 2, the active component 42 comprises one transistor 43. The transistor is for example a FET-transistor, preferably a thin film transistor (TFT) as known from active matrix liquid crystal displays, having its gate terminal connected to the specific selection line 61 and having its source/drain terminal connected to the specific signal line 71.

Preferably, the thin film transistor is fabricated from any of the well known active matrix technologies as known from manufacturing of active matrix liquid crystal displays and other active matrix displays. These technologies include the amorphous silicon (a-Si) technology, low temperature poly silicon technology (LTPS), nanocrystalline Si technology, microcrystalline Si technology, CdSe technology, SnO technology, polymer or organic semiconductor based technology etc. In some cases only transistors of one polarity are available (e.g. a-Si provides only N-type transistors), while in other cases transistors of both polarities are available (e.g. LTPS provides n-type and p-type transistors). If an appropriate voltage level is applied to the specific selection line 61, the transistor switch will become conductive and thereby electrically connect the specific signal line 71 to the first electrode 40 (connected to the drain/source terminal of the transistor 43) of the compartment 20 in the middle of the matrix arrangement of compartments depicted in FIG. 2. This means, that the release mechanism 30 would be removed or activated by applying an appropriate electrical signal to the specific signal line 71. Of course, the function of the selection and signal lines 60, 70 (or the specific selection and signal lines 61, 71) can also be inverted, i.e. the signal lines 70 are connected to the gate terminal of the transistor 43 and the selection lines 60 are connected to the source/drain terminal of the transistor 43. Of course—as depicted in FIG. 2—each compartment 20 is equipped with one transistor 43. The second electrodes 50 of each of the compartments 20 are connected to a further electrically conductive line in common or in groups. For the sake of clarity, this further electrically conductive line is not shown in FIG. 2.

For the sake of clarity the release mechanism 30 is not depicted in FIG. 2. In contrast, a first driver 65 (also called select driver 65) for driving the selection lines 60 is shown in FIG. 2 as well as a second driver 75 (also called central driver 75) for driving the signal lines 70. In the special example of FIG. 2, the gate terminals of the transistors 43 are connected to the select driver 65 (which can be provided as a standard shift register gate driver as used for an active matrix liquid crystal display) and the source terminals of the transistor 43 are connected to the central driver 75. Furthermore, a control unit 80 for controlling the release of the substance is also shown in FIG. 2. The control unit 80 controls the first and the second driver 65, 75 for defining, by means of specific selection lines 61, 71, a specific compartment 20. The control unit 80 also controls the successive activation of different compartments 20. This means, that the control unit 80 for example controls the opening of the release mechanisms 30 of different compartments such that for example the concentration of a drug remains at an optimum therapeutic level during the course of a treatment. As the optimum concentration of the drug is variable from one patient to the next, and during the course of the treatment it is necessary that this drug delivery system be extremely flexible and provide an almost continuously variable dosage of the drug. Such a drug release system is possible to realize with the inventive device. Preferably, the control unit 80 either has sensors for determining the actual level of the drug in the environment of the device 10 or the device 10 is coupled to such a sensor device (not shown) so that a signal from the sensor device signalling the control unit 80 to increase or decrease drug release results in an appropriate reaction of the inventive device, i.e. the control unit 80 activates the first and second drivers 65, 75 in order to increase or decrease the release of the substance inside the compartments 20.

For example, if the drug delivery i.e. the opening of the release mechanisms 30 is based upon an electro-chemical reaction which breaks the seal of the compartment 20 or which breaks the release mechanism 30 of the compartment 20, and where a voltage of around 1 V is required to initiate the electro-chemical reaction, it is possible to use a standard voltage data driver as used for e.g. active matrix liquid crystal displays. For example, either of the first and second electrodes is provided as a cathode and the other electrode of the first and second electrodes serves as an anode. The anode is defined as the electrode where oxidation occurs. Any conductive material capable of dissolving into solution or forming soluble ions or oxidation compounds upon application of an electric current or an electric potential (electrochemical dissolution) can be used for the fabrication of the anodes and cathodes. In addition, materials that normally form insoluble ions of oxidation products in response to an electric potential can be used if, for example, local pH changes near the anode cause these oxidation products to become soluble. Examples of suitable reservoir cap materials include metals such as copper, gold, silver, and zinc, and some polymers.

The inventive device 10 in the example shown in FIG. 2 works as follows: In the state of rest, all selection lines 60 (also called select lines 60) are set to a voltage where the transistors 43 are non-conducting. In this case no release mechanism 30 is opened and therefore no substance or drug released. To release a substance or a drug out of one compartment 20, the transistors in the entire row of compartments 20 including the required compartment are switched into the conducting state (by e.g. applying a positive voltage). The voltage in the column where the compartment 20 to be activated is located is set to its release voltage (e.g. 1 V). This voltage is passed through the conducting thin film transistor to either of the first and second electrodes 40, 50 of the selected compartment 20, resulting in drug release. The voltage in all other columns is held at a voltage that will not release the drug (this will be typically 0 V). After the drug is released, the transistors 43 in the selected row are again set to the non-conducting state, preventing further drug release.

It is also possible to release drugs or a substance or substances from more than one compartment 20 in a given line (or in a given row) simultaneously by applying a release signal (preferably a voltage) to more than one column in the array. It is possible to sequentially release drugs from compartments 20 in different rows by activating another one of the selection lines 60 (using the select driver 65) and applying a release signal (preferably a voltage) to one or more column selection lines 70 in the array. The specific compartment 20 which is selected by the specific selection line 61 and the specific signal line 71 in FIG. 2 is located in the middle of the matrix arrangement of compartments 20. If the transistor 43 is conducting and the specific signal line activated, the voltage between the first and second electrodes 40, 50 of the selected compartment 20 is then amounting for example to 1 V, thus initiating the drug release. The voltage in the other compartments 20 is held at a level at which the drug will not be released. After the drug or the substance has been released, the selection line 60 and the signal line 70 are again set to 0 V, which corresponds also to the state of rest of the inventive device 10, thereby saving electrical power.

In one embodiment of the present invention it is also possible to release a drug or a substance from more than one compartment in a given row simultaneously by applying a release signal to more than one row, i.e. more than one specific selection line 61 in the array. Then different compartments 20 are simultaneously selected as being active, i.e. as being opened through removing the release mechanism 30 or by disintegrating the release mechanism 30. Accordingly it is also possible to simultaneously or sequentially release drugs from compartments 20 in different columns by activating a specific selection line 61 and applying a release signal to one or more columns in the array.

In another embodiment of the present invention, the drug delivery mechanism, i.e. the mechanism for opening the release mechanism 30, is based upon a heating effect, i.e. the heating of the release mechanism 30 breaks the release mechanism 30 of the compartment 20 which is selected. In this case, electrodes 40, 50 are electrically connected via the heating element, which could be any one of the known heating elements such as a resistive heater, peltier element etc.

When the release mechanism, i.e. the opening mechanism of the release mechanism 30, is provided as an electro-chemical reaction, the first or second electrode 40, 50 can for example be provided as a gold layer in the vicinity of the release mechanism 30. The other one of the first and/or second electrode 40, 50 is for example another metallized electrode connected in common. By applying a voltage between the first and second electrodes 40, 50 a gold layer or gold cap acts as an anode in an electrochemical reaction and is resolved when a sufficiently high voltage is applied. When the gold layer or the gold cap is removed, then either the closure cap 30 is also removed because the closure caps 30 consists essentially of the gold cap, or the removing of the gold cap sufficiently weakens the closure cap 30 made of another material such that the closure cap 30 will break if the gold cap is removed. Anyway, after the electro-chemical reaction has taken place, the substance or drug inside the compartment 20 is freed and allowed to diffuse away. In such an embodiment of the inventive device, the substrate 11 is for example provided in the form of a silicon wafer containing the compartments 20 as micro reservoirs which are etched into the silicon substrate.

According to a feature of any of the described embodiments of the present invention, the substrate 11 or the chip can be packaged with a battery and a micro processor or a control unit to be completely self-contained. Preferably the control unit 80 is monolithically integrated with the substrate 11 having the compartments 20.

The compartment 20 contents comprise essentially any object or material that needs to be isolated (e.g. protected from) the environment outside the compartment 20 until a selected point in time, when its release or exposure is desired. In various embodiments, the compartment 20 contents comprise a certain quantity of molecules or of a specific substance or of a mixture of specific substances. Proper functioning of certain reservoir contents such as a catalyst or a sensor generally does not require the release of the compartment contents. Rather, their intended function, e.g. catalyzing or sensing, occurs upon exposure of the reservoir contents to the environment outside of the compartment 20 after opening of the closure cap 30. Thus, the catalyst molecules or sensing component can be released or can remain immobilised within the open compartment 20. Other compartment contents such as drug molecules may often need to be released from the compartment in order to pass from the device and be delivered to a site in vivo to exert a therapeutic effect on a patient. However, the drug molecules may be retained for certain in-vitro applications. The compartment 20 contents can include essentially any natural or synthetic, organic or inorganic molecule or mixture thereof. The molecules may be in essentially any form, such as a pure solid or liquid, a gel or hydrogel, a solution or emulsion, a slurry or a suspension. The molecules of interest may be mixed with other materials to control or enhance the rate and/or time of release of an open compartment 20. In various embodiments, the molecules may be in the form of solid mixtures, including amorphous or crystalline mixed powders, monolithic solid mixtures, lyophilized powders and solid interpenetrating networks. In other embodiments, the molecules are in liquid forms, such as solutions, emulsions, colloidal suspensions, slurries or gel-mixtures such as hydrogels.

In FIG. 3 an inventive device 10 according to a further embodiment is schematically shown. In contrast to the device 10 according to the embodiment of FIG. 2, the device 10 of FIG. 3 is either (first alternative of the device of FIG. 3) not capable of providing (if required) different (independent) signals (e.g. different voltages) to a multitude of signal lines 70 or (second alternative of the device of FIG. 3) the device 10 of FIG. 3 is not capable of providing (if required) signals to a multitude of columns selection lines 70 of the array simultaneously. The advantage of the device 10 depicted in FIG. 3 is that a much simpler second driver 75 can be used. Such a simpler second driver 75 has the function of a de-multiplexer. In the embodiment of the device 10 depicted in FIG. 3, only a single output driver 76 is required to generate the release signal (e.g. a voltage). The function of the de-multiplex circuit in the first alternative of the device of FIG. 3 is to route one identical release signal to one or a plurality of the multitude of signal lines 70. The function of the de-multiplex circuit in the second alternative of the device of FIG. 3 is to route the release signal to only one from the multitude of signal lines 70, whereby only the drug or substance is released in the compartment in the selected row in that column. The de-multiplex circuit has the effect of closing one or a plurality of switches 77 at the appropriate column (first alternative of FIG. 3) or of closing one simple switch 77 at the appropriate column (second alternative of FIG. 3).

In FIG. 4 an embodiment of a compartment 20 with an active component 42 is schematically shown. For the sake of simplicity, only one compartment 20 together with the active component 42 is shown. Of course, the corresponding inventive device 10 would comprise a matrix arrangement of compartments 20 of this type. The active component 42 comprises a first transistor 43 and a second transistor 44 and a power voltage line 46. The compartment 20 again comprises the first electrode 40 and the second electrode 50. The device 10 comprises the selection lines 60 and the signal lines 70 together with the appropriate drivers 65, 75. The embodiment of FIG. 4 is especially preferable if not only a simple voltage signal is to be used as a release signal but a different release signal, e.g. a current adjusted release signal for example to be applied to a heating element. If a release current (i.e. the proper functioning of the release is determined by the proper application of a signal with a precise current flowing) is required, it can be difficult to realize a driver means 65, 75 cost-effectively, which produces a controlled current. Furthermore, it is not always possible to bring the needed current or the correct current from the driver means 65, 75 to the compartment electrode 40, 50 without a loss of current, due e.g. to electrical leakage effects. For both reasons, it is possible according to the invention to use the active matrix arrangement of the device to create a separate release driver per compartment as a part of the active component 42, thereby being able to create locally the release signal. In FIG. 4, such a local release driver as a part of the active component in each compartment comprises not only a select transistor 43 (first transistor 43) but also a local current source. This local current source can be realized by means of the second transistor 44. The current flowing through this second transistor 44 is defined by the voltage (also called V_{gate}) at the gate terminal (reference sign 47), i.e. the transconductance of the transistor (the current through the transistor is a constant multiplied by the squared term of (V_{power}−V_{gate}−Vₜ). The programming of the release signal is provided by a specified voltage from the voltage driver via the select transistor (first transistor 43) to the gate terminal 47 of the second transistor 44.

In FIG. 5, further embodiments of compartments 20 with active components 42 comprising a memory element 45 or memory means 45 are schematically shown. For the sake of simplicity, a first embodiment of such a compartment 20 with a memory element 45 is shown in FIG. 5, left side and a second embodiment of such a compartment 20 with a memory element 45 is shown in FIG. 5, right side. Of course it is also clear that the preferred device 10 according to the present invention comprises compartment 20 with active components 42 of the same type for all the components or for the whole matrix arrangement of the device 10. In FIG. 5 left hand side, the active component 42 with the first transistor 43 according to the embodiment of FIG. 2 is shown where only the memory element 45 or memory means 45 is added, e.g. in the form of a capacitor having its first terminal connected to the first electrode 40 and having its second terminal connected to the second electrode 50. The memory element 45 or memory means 45 allows for an extension in time of the drive signal beyond the time that the selected compartment 20 is (electrically) addressed. In this example, the memory element 45 is a simple capacitor parallel to the capacitance formed by the compartment electrodes (first and second electrodes 40, 50). This capacitor stores the voltage and maintains it while e.g. another line (or row) of compartments 20 is being addressed.

In the case of a current signal needed (e.g. realized by the embodiment according to FIG. 4), the extra capacitor (see FIG. 5 right hand side) is situated to store the voltage on the gate terminal of the current source (second transistor 44) and maintain the release current while e.g. another line (or row) of compartments is being addressed.

Generally, adding the memory element 45 (which could also be realized by means of another impedance element such as an inductance, or alternatively a transistor-based memory circuit such as an inverter, flip-flop or RAM) allows the release signal to be applied for a longer period of time, whereby the drug release may be carried out more reliably and/or more quickly.

According to the present invention, other local release drivers are possible, e.g. local oscillators or other drivers to generate pulse waveforms or the like.

In FIG. 6, further embodiments of compartments 20 with active components 42 are schematically shown where the active matrix device according to the present invention is based on diodes. For the sake of simplicity, in FIG. 6 a first embodiment of such a compartment 20 (single-diode concept) is shown in FIG. 6, left side, a second embodiment of such a compartment 20 (parallel diodes concept) is shown in the middle of FIG. 6 and a third embodiment of such a compartment 20 (series connected concept) is shown in FIG. 6, right side. While offering somewhat less flexibility than using TFTs, it is also possible to realize an active matrix-controlled release device using the technologically less demanding thin film diode technology. Diode active matrix arrays (as have been used for e.g. active matrix LCDs) can be driven in several known ways, one of which is the double diode with reset (D2R) approach, see K. E. Kuijk, Proceedings of the 10^{th} International Display Research Conference (1990, Amsterdam), p 174. which is incorporated herein by reference.

The circuit for driving or for activating one compartment of these diode embodiments of an active matrix array is shown in FIG. 6.

The diode matrix in the first embodiment (FIG. 6, left side) has two diodes D1, D2 per compartment 20, one (D1) to deliver the signal to the compartment 20 via the signal line 70 and one (D2) to remove the signal from the compartment via a common reset line 72. The blocking range, that is the voltage range where the diodes D1, D2 are non-conducting, is determined by the external voltages and therefore adjustable. This is a major advantage where higher operating voltage components are required. Higher voltages can easily be accommodated by providing diodes in series (see the third embodiment of FIG. 6, right hand side: diodes D5 as well as diodes D6 comprise two individual diode elements) as this prevents breakdown of separate diodes at high reverse voltage—the voltage is split across the diodes. The number of external connections in all three embodiments of FIG. 6 is equal to the number of rows (or selection lines 60) plus the number of columns (or signal lines 70) plus one (the reset line 72). The circuit is very independent of the diode characteristics and both pin or Schottky diodes can be chosen. The circuit can be made redundant for short or open circuit errors by using extra diodes in series (FIG. 6 right hand side) or in parallel (FIG. 6 in the middle). The rows are driven for example by using a reset method with five voltage levels as described in K. E. Kuijk, Proceedings of the 10^{th} International Display Research Conference (1990, Amsterdam), p 174, which is incorporated herein by reference.

A PIN (or Schottky-IN) diode can be formed using a simple 3-layer process. An amorphous semiconductor layer, a stack of p-doped, intrinsic, and n-doped regions, is sandwiched between top and bottom metal lines, which are oriented perpendicularly. The electrical properties are hardly sensitive to alignment.

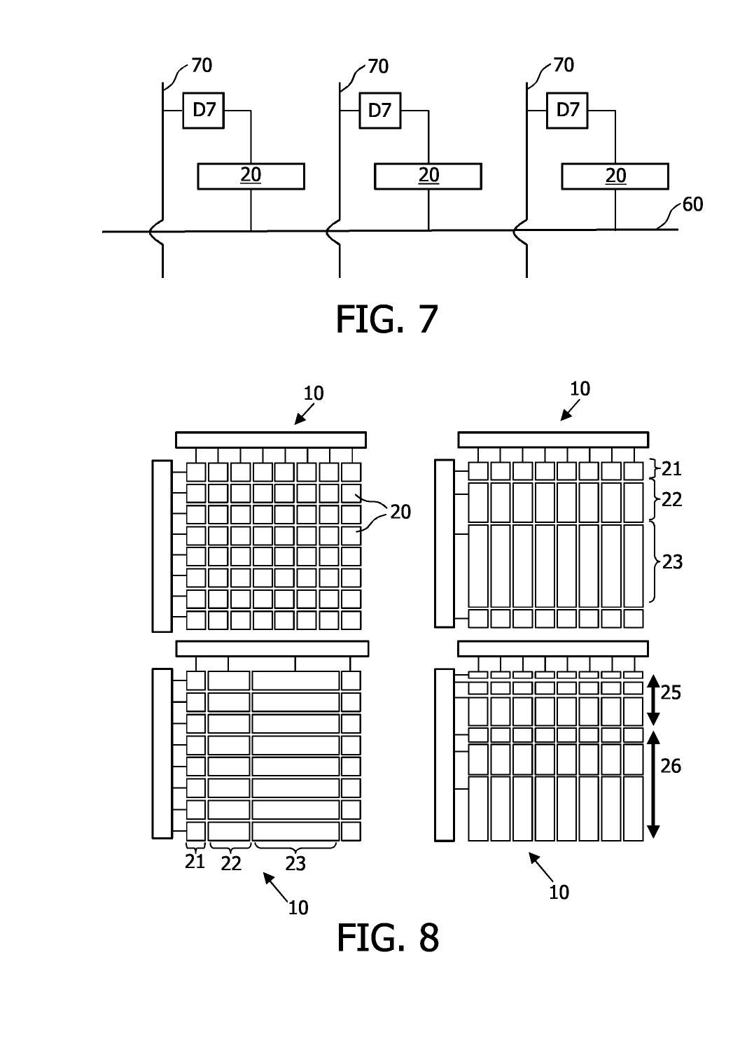

In FIG. 7, a further embodiment of compartments 20 with active components 42 is schematically shown where the active matrix device according to the present invention is based on MIM-diodes. While offering somewhat less flexibility than using TFTs, it is also possible to realize an active matrix-controlled release device using the technologically less demanding metal-insulator-metal (MIM) diode technology.

Traditionally, MIM diode active matrix arrays (as are used for active matrix LCDs) have a layout similar to a passive matrix. However, a MIM diode is introduced as a non-linear resistance element in series with each compartment 20, to allow for active matrix addressing. The compartment 20 has two electrodes (first and second electrodes, not shown in FIG. 6 for the sake of simplicity) in the example of FIG. 6. A MIM diode D7 is introduced as a non-linear element connecting the signal line 70 to the compartment 20. The MIM device (or MIM-Diode) D7 is created by separating two metal layers by a thin insulating layer (examples are hydrogenated silicon nitride sandwiched between Cr of Mo metals, or Ta₂O₅ insulator between Ta metal electrodes, see e.g. A. G. Knapp and R. A. Hartman, Proc 14^{th} Int Display Research Conf (1994) p. 14 as well as S. Aomori et al, SID 01 Digest (2001) p. 558. These disclosures are incorporated herein by reference.), and is conveniently realized in the form of a cross-over structure. The MIM connects the selection line 60 (shown) or the signal line (shown) to either the first or second electrode 40, 50 (not shown for the sake of simplicity) of the compartment 20. Both metal layers and also the insulation layer are realized on the same substrate. In FIG. 7, the connections of the compartment 20 are completed by adding the selection line 60 providing the select signal.

In FIG. 8 four different arrangements of compartments 20 within an inventive device 10 are schematically depicted. In a first embodiment of the device 10 (see FIG. 8 top left) all the compartments 20 are of the same size and provided in a matrix arrangement. The size of the compartments 20 defines a first quantity of a substance contained in the compartments 20. It is either possible that all compartments 20 contain the same substance or it is possible that in a first group (not shown) of the compartments 20, a first substance is located and that in a second group (not shown) of the compartments 20 a second substance is located.

In the second example shown in FIG. 8 (see FIG. 8 top right) an inventive device 10 is depicted where a first group 21 of compartments 20 has a predefined size, allowing to contain a first quantity of a substance. A second group 22 of compartments 20 comprises compartments 20 which are larger than the compartments 20 of the first group 21. Thus, the compartments of the second group 22 are for example able to contain a second quantity of a substance which is twice the first quantity. Of course any other ratio of the first and second quantities is also possible. A third group 23 of compartments 20 comprises compartments 20 which are able to contain a third quantity of a substance. The third quantity being for example twice the second quantity and four times the first quantity. Of course the third quantity can also be provided in a different ratio regarding the first and second quantity. By selecting specific compartments 20 from the first, the second or the third group 21, 22, 23 of compartments 20 it is possible according to the present invention to release a higher or lower amount or quantity of a substance from the compartments 20 by means of just opening one single compartment 20. This has the advantage that the release of different quantities of the substance can be controlled very easily and with small efforts especially with respect to the control unit 80.

In a third example of the inventive device 10 of the present invention depicted in FIG. 8 (see FIG. 8 bottom left) a matrix arrangement of compartments 20 with different groups 21, 22, 23 of compartments 20 is shown. In the third example the arrangement of compartments 20 is comparable to the arrangement of compartments 20 in the second example (FIG. 8 top right). In the third example the size of compartments in each row of the matrix arrangement is identical, whereas the different groups of compartments are realized by changing the size of compartments 20 between different columns. In contrast, in the second example (see FIG. 8 top right) the compartments of each column are identically sized and the compartments of different rows are different.

In a fourth example of a matrix arrangement of the compartments 20 in an inventive device 10 according to the present invention, a first area 25 of compartments 20 is defined, which contains a first substance and a second area 26 of compartments 20 is defined, which contains a second substance.

By the examples given of different matrix arrangements of the compartments 20 of an inventive device, it is possible to have a high flexibility in dosing different quantities and/or different substances by means of the inventive device 10. By changing the size of the compartments 20 and hence the quantities of substances released, a more flexible drug delivery is possible with a smaller number of compartments. For example by providing compartments of sizes in the range of 1:2:4:8:16 etc. it is possible to provide a wide range of dosing a simultaneously opening of one or more compartments 20 in a controlled manner. In the case of the delivery of more than one type of substance (see example four of FIG. 8, bottom right) it is usual for different drugs to have different dosing quantities. For this reason it will be preferred to have different sections or areas 25, 26 of the matrix array of compartments 20, with proportionally larger or smaller compartments 20 depending upon the drug to be delivered. This is preferably achieved by uniformly increasing this spacing between selection line 60 and/or signal line 70 in the array as is illustrated in FIG. 8 (bottom right), as this makes the best use of the available drivers 65, 75, and to reduce redundancies of elements included in the device 10. Depending on the complexity of the desired device 10, a memory or shift register is needed to keep the status of the compartments 20 used and those still available updated. Such a memory device can advantageously be included in the control unit 80 of the device 10.

1. Device (10) for the controlled release of a predefined quantity of a substance, the device (10) comprising a matrix arrangement of compartments (20) in a substrate (11), each compartment (20) being closed by at least one release mechanism (30), at least one first electrode (40) and at least one second electrode (50) being assigned to each compartment (20), the device (10) comprising a plurality of selection lines (60) and a plurality of signal lines (70), the number of compartments (20) exceeding the sum of the number of selection lines (60) and the number of signal lines (70), each first electrode (40) or each second electrode (50) being electrically connected via at least one active component (42) to one of the plurality of selection lines (60) and/or to one of the plurality of signal lines (70).

2. Device (10) according to claim 1, wherein the release mechanism (30) is a one-time release mechanism (30).

3. Device (10) according to claim 1, wherein the release mechanism (30) is a closure cap.

4. Device (10) according to claim 1, wherein each compartment (20) is defined by means of one specific selection line (61) from the plurality of selection lines (60) and one specific signal line (71) from the plurality of signal lines (70).

5. Device (10) according to claim 1, wherein the number of compartments (20) is in the order of magnitude of the number of selection lines (60) multiplied by the number of signal lines (70).

6. Device (10) according to claim 5, wherein the number of selection lines substantially equals the number of signal lines.

7. Device (10) according to claim 1, wherein the active component (42) comprises a transistor (43) assigned to each compartment (20).

8. Device (10) according to claim 7, wherein the transistor (43) is a thin-film transistor.

9. Device (10) according to claim 7, wherein the gate of the transistor (43) is connected to a selection line (60) and wherein the main conducting channel connects a signal line (70) to either a first or a second electrode (40, 50).

10. Device (10) according to claim 1, wherein the active component (42) comprises a first transistor (43) and a second transistor (44).

11. Device (10) according to claim 1, wherein the active component (42) comprises a local current source.

12. Device (10) according to claim 1, wherein the active component (42) comprises a diode.

13. Device (10) according to claim 1, wherein the active component (42) comprises an MIM diode.

14. Device (10) according to claim 1, wherein the active component (42) comprises a memory means (45).

15. Device (10) according to claim 1, wherein a first group (21) of compartments (20) is provided to contain a first quantity of a first substance and a second group (22) of compartments (20) is provided to contain a second quantity of a second substance.

16. Device (10) according to claim 15, wherein the first quantity is approximately half the second quantity.

17. Device (10) according to claim 1, wherein the release mechanism (30) of the compartment (20) is operated by applying an electrical potential between the first electrode (40) and the second electrode (50).

18. Device (10) according to claim 1, wherein the first electrode (40) and the second electrode (50) of each compartment (20) are electrically insulated from each other.

19. Device (10) according to claim 1, wherein the release mechanism (30) is activated by means of an electrochemical reaction.

20. Device (10) according to claim 1, wherein the release mechanism (30) is activated by means of heating the release mechanism (30).

21. Device (10) according to claim 1, wherein the device (10) comprises a control unit (80) for controlling the release of the substance.

22. Device (10) according to claim 1, wherein the number of compartments (20) is at least 100.

23. Device (10) according to claim 1, comprising materials from the group of LTPS (low temperature polycrystalline silicon), amorphous silicon, nanocrystalline Si, microcrystalline Si, or other semiconducting material such as CdSe, SnO or organic semiconductors.

24. Method for controllably releasing a predefined quantity of a substance from a compartment (20), using a device (10) comprising a matrix arrangement of compartments (20) in a substrate (11), each compartment (20) being closed by at least one release mechanism (30), at least one first electrode (40) and at least one second electrode (50) being assigned to each compartment (20), the device (10) comprising a plurality of selection lines (60) and a plurality of signal lines (70), the number of compartments (20) exceeding the sum of the number of selection lines (60) and the number of signal lines (70), the method comprising the steps of:
electrically connecting each first electrode (40) or each second electrode (50) via at least one active component (42) to one of the plurality of selection lines (60) and/or to one of the plurality of signal lines (70),
activating the active component (42) and thereby applying an electrical potential or a current between the first electrode (40) and the second electrode (50).

25. Method according to claim 24, wherein more than one compartment (20) release the substance at the same time.
